(19) United States
(12) Patent Application Publication
(10) Pub. No.: US 2025/0196188 A1
(43) Pub. Date: Jun. 19, 2025

(54) MEDICAL INSTRUMENT WITH A PIEZOELECTRIC SENSOR STACK, AND METHOD FOR PRODUCING SAME

(71) Applicant: B. Braun Melsungen AG, Melsungen (DE)

(72) Inventors: Nikolaus Berger-Roscher, Kassel (DE); Daniel Baasner, Minden (DE); Eike Kottkamp, Luebbecke (DE); Walter Wirths, Taufkirchen (DE)

(21) Appl. No.: 18/841,012

(22) PCT Filed: Feb. 8, 2023
(86) PCT No.: PCT/EP2023/053112

§ 371 Date: Aug. 23, 2024

(30) Foreign Application Priority Data
Feb. 25, 2022 (DE) 10 2022 201 953.9

(51) Int. Cl. B06B 1/06; A61B 8/08; A61B 8/12

(52) U.S. Cl. CPC B06B 1/0688; A61B 8/0841; A61B 8/12; B06B 2201/76

(57) ABSTRACT

A medical instrument has a distal end for insertion into a patient and a sensor unit at the distal end that is configured to capture and convert a measured physical variable into an electrical measurement signal. The sensor unit is a sensor stack constructed in layers and having a first portion and a second portion. The first and second portions each have functional layers arranged one above another. The functional layers are applied to one another in layers and cover one another at least in portions. The first portion includes a first insulation layer, a first electrode, a piezoactive piezo layer, a second electrode layer, a second insulation layer, and a shielding layer. The second portion includes an insulation layer, a first conductor track, and a second conductor track. The sensor unit and the medical instrument are produced by a method.

### CROSS-REFERENCE TO RELATED APPLICATIONS

This application is the United States national stage entry of International Application No. PCT/EP2023/053112, filed on Feb. 8, 2023, and claims priority to German Application No. 10 2022 201 953.9, filed on Feb. 25, 2022. The contents of International Application No. PCT/EP2023/053112 and German Application No. 10 2022 201 953.9 are incorporated by reference herein in their entireties.

### FIELD

The invention relates to a medical instrument having a sensor stack and to a method for producing such a medical instrument.

### BACKGROUND

A medical instrument in the form of a catheter with a sensor unit for capturing and converting a measured physical variable is known from WO 2017/013224 A1, for example. The sensor unit is embodied in the form of an ultrasonic transducer and serves to locate the tip of the catheter in the body of a patient on the basis of ultrasound, which is also referred to as tracking. The sensor unit is embodied from a plurality of film-like functional layers layered on one another and can therefore also be referred to as a sensor stack. In this context, the sensor stack is manufactured by adhesively bonding the individual functional layers to one another layer-by-layer, which is also referred to as laminating. For application purposes, the laminated sensor stack is provided with an adhesive layer, wound around the catheter tip, and adhesively bonded to the catheter tip by means of the adhesive layer.

Further, DE 10 2018 220 606 A1 has disclosed a medical instrument, possibly designed as a cannula for example, which at a distal end comprises a sensor unit configured to capture a measured physical variable and convert the latter into an electrical measurement signal.

A frequent disadvantage of state-of-the-art medical instruments with a sensor unit lies in an inadequate capture of measured variables and consequently a low signal strength, whereby navigating the medical instrument in the body of a patient to a target area, for example, is made more difficult. Moreover, as a rule, the production of correspondingly designed medical instruments is very time-consuming and expensive.

### SUMMARY

The object of the invention therefore lies in the provision of a medical instrument and a method for producing a medical instrument, which in particular avoid the disadvantages mentioned at the outset in part or in full.

According to a first aspect, the invention relates to a medical instrument having a distal end which is provided for insertion into the body of a patient. In particular, the medical instrument can be a catheter, a cannula or any other medical instrument provided for at least partial insertion into the body of a patient. Accordingly, the distal end can be a catheter tip or a cannula tip in particular.

The medical instrument comprises a sensor unit which is arranged at the distal end. The sensor unit is configured to capture a measured physical variable and convert the latter into an electrical measurement signal, said sensor unit being in the form of a sensor stack constructed in layers. For example, the sensor stack can be configured to capture a temperature, a hydrostatic pressure or any other measured physical variable that is of therapeutic and/or diagnostic interest. By preference, the sensor stack, in particular the piezo layer of the first portion, is configured to capture and convert ultrasonic pulses, thus preferably forming an ultrasonic transducer.

The sensor stack comprises a first portion and a second portion. The first portion can also be referred to as sensor portion within the meaning of the present invention. The second portion can also be referred to as contacting portion within the meaning of the present invention; there, electrical signals arising in the first portion of the sensor stack can be tapped or conducted away.

By preference, the first portion is a distal portion of the sensor stack, and the second portion is a proximal portion of the sensor stack. In other words, the sensor stack can be arranged on the medical instrument in such a way that the first portion is arranged at the distal end of the medical instrument in the distal direction, and the second portion is arranged at the distal end of the medical instrument in the proximal direction.

Further, the first portion and the second portion are preferably arranged in succession, in particular in direct succession. The first portion and the second portion are particularly preferably embodied contiguously in one piece. Alternatively, the first portion and the second portion can be embodied separately from one another and can be connected, in particular integrally bonded, to one another.

In particular, the sensor stack can consist of the first portion and the second portion.

By preference, the sensor stack is produced by means of a printing method, in particular offset printing, inkjet printing, screen printing, aerosol jet printing, spray coating or dip coating.

The first portion and the second portion of the sensor stack each comprise a plurality of functional layers arranged one above the other, in particular arranged in a manner directly or not directly above another. The functional layers are applied to, in particular printed on, one another in layers so as to cover one another at least in portions, in particular only in portions or in full.

The first portion comprises at least the following functional layers:
a first insulation layer covering and electrically insulating the distal end of the medical instrument at least in portions, in particular only in portions or in full, i.e. throughout or contiguously,
an electrically conductive first electrode layer applied to, in particular printed on, the first insulation layer at least in portions, in particular only in portions or in full, i.e. throughout or contiguously,
a piezoactive piezo layer applied to, in particular printed on, the first electrode layer at least in portions, in particular only in portions or in full, i.e. throughout or contiguously,
an electrically conductive second electrode layer applied to, in particular printed on, the piezo layer at least in portions, in particular only in portions or in full, i.e. throughout or contiguously,
an electrically insulating second insulation layer applied to, in particular printed on, the second electrode layer at least in portions, in particular only in portions or in full, i.e. throughout or contiguously, and
a shielding layer applied to, in particular printed on, the second insulation layer at least in portions, in particular only in portions or in full, i.e. throughout or contiguously.

In this case, the first insulation layer can directly or not directly cover the distal end of the medical instrument.

Further, the first electrode layer can be directly or not directly applied to, in particular printed on, the first insulation layer and/or the piezo layer can be directly or not directly applied to, in particular printed on, the first electrode layer and/or the second electrode layer can be directly or not directly applied to, in particular printed on, the piezo layer and/or the second insulation layer can be directly or not directly applied to, in particular printed on, the second electrode layer and/or the shielding layer can be directly or not directly applied to, in particular printed on, the second insulation layer.

The second portion comprises at least the following functional layers:
an insulation layer covering and electrically insulating the distal end of the medical instrument at least in portions, in particular only in portions or in full, i.e. throughout or contiguously,
an electrically conductive first conductor track applied to, in particular printed on, the insulation layer at least in portions, in particular only in portions or in full, i.e. throughout or contiguously, and
an electrically conductive second conductor track applied to, in particular printed on, the insulation layer at least in portions, in particular only in portions or in full, i.e. throughout or contiguously.

In this case, the insulation layer can directly or not directly cover the distal end of the medical instrument.

Further, the first conductor track can be directly or not directly applied to, in particular printed on, the insulation layer and/or the second conductor track can be directly or not directly applied to, in particular printed on, the electrically insulating insulation layer. By preference, the first conductor track and the second conductor track are applied to, in particular printed on, different portions of the insulation layer.

Within the meaning of the present invention, the term “distal” should be understood to mean situated further away from the body center of a person, in particular a physician, who is using the medical instrument as intended.

Within the meaning of the present invention, the term “proximal” should be understood to mean situated closer to the body center of a person, in particular a physician, who is using the medical instrument as intended.

Within the meaning of the present invention, the term “sensor stack” should be understood to mean a sensor unit constructed layer-by-layer from a plurality of functional layers and embodied as film or foil in particular.

Within the meaning of the present invention, the term “piezoactive piezo layer” should be understood to mean a layer where a voltage is created under the action of mechanical pressure (piezoelectric effect).

The shielding layer preferably serves to shield the remaining functional layers, in particular the first portion, preferably the piezo layer, of the sensor stack from electric and/or magnetic fields. Such fields can lead to an error when capturing measured variables and/or converting measured variables and/or conducting away measured variables. The shielding layer enables a sensor stack function that is as error-free as possible.

The first conductor track and the second conductor track of the second portion serve to conduct away the electrical measurement signals from the first portion, in particular the piezo layer of the first portion, in the direction of a proximal end of the medical instrument. Proceeding from this proximal end, the first conductor track and the second conductor track can be electrically conductively contacted or contactable with an evaluation unit for the purpose of evaluating the electrical measurement signals from the first portion, in particular the piezo layer of the first portion.

Further preferably, the first insulation layer of the first portion and the insulation layer of the second portion have the same embodiment. In particular, the first insulation layer of the first portion and the insulation layer of the second portion are embodied contiguously in one piece. Alternatively, the first insulation layer of the first portion and the insulation layer of the second portion can be embodied separately from one another and can be connected, in particular integrally bonded, to one another.

The second portion of the sensor stack is preferably devoid of a further insulation layer, in particular a further insulation layer applied to, in particular printed on, the first conductor track and/or second conductor track, and devoid of an electrically-shielding shielding layer. In other words, the first conductor track and the second conductor track of the second portion are each by preference an exposed conductor track. Advantageously, this can bring about contacting with, for example, an amplifier unit configured to amplify the measurement signals from the sensor stack and/or with an evaluation unit disposed downstream of the amplifier unit in particular and configured to evaluate the electrical measurement signals.

Further preferably, the first electrode layer of the first portion and the first conductor track of the second portion are embodied contiguously in one piece. A one-piece embodiment of the first electrode layer and first conductor track is advantageous in that, in principle, electrical signals arising in the first portion of the sensor stack can be tapped more quickly.

Alternatively, the first electrode layer can be applied, in particular printed on, separately from the first conductor track and electrically contacted with the latter.

Further, the second electrode layer of the first portion and the second conductor track of the second portion are preferably embodied contiguously in one piece. A one-piece embodiment of the second electrode layer and second conductor track is (likewise) advantageous in that, in principle, electrical signals arising in the first portion of the sensor stack can be tapped more quickly.

Alternatively, the second electrode layer can be applied, in particular printed on, separately from the second conductor track and electrically contacted with the latter.

The functional layers of the first portion and/or second portion independently of one another can have a layer thickness of 1 μm to 30 μm, in particular 0.5 μm to 20 μm, and by preference 1 μm to 10 μm.

In a configuration of the invention, the first electrode layer and the second electrode layer are embodied differently, in particular at least in portions, for example only in portions or throughout.

Alternatively, the first electrode layer and the second electrode layer can be embodied the same, in particular at least in portions, for example only in portions or throughout.

Further, the first electrode layer, in particular only the first electrode layer, can be embodied contiguously in one piece or in the form of layer portions which are embodied separately from one another. Further, the second electrode layer, in particular only the second electrode layer, can be embodied contiguously in one piece or in the form of layer portions which are embodied separately from one another.

Further, the first electrode layer can be embodied contiguously in one piece, and the second electrode layer can be embodied in the form of layer portions which are embodied separately from one another. Alternatively, the first electrode layer can be embodied in the form of layer portions which are embodied separately from one another, and the second electrode layer can be embodied contiguously in one piece. Alternatively, the first electrode layer and the second electrode layer can each be embodied contiguously in one piece or each be embodied in the form of layer portions which are embodied separately from one another.

Further, the first electrode layer can have a layer thickness of 0.1 μm to 30 μm, in particular 0.5 μm to 10 μm, and by preference 1 μm to 2 μm. The second electrode layer can further have a layer thickness of 0.1 μm to 30 μm, in particular 0.5 μm to 10 μm, and by preference 1 μm to 2 μm.

In principle, any electrically conductive material is suitable for the first electrode layer and the second electrode layer.

By preference, the first electrode layer and the second electrode layer each comprise a printable conductive material or preferably each consist of such a material. In particular, at least one of the two electrode layers comprises a material or consists of a material that can be structured very finely, in particular be structured in the micrometer range, for example be structured with a line width of 100 μm.

Further preferably, the first electrode layer and the second electrode layer independently of one another comprise an electrically conductive material or consist of an electrically conductive material, the electrically conductive material being selected from the group consisting of electrically conductive polymers, electrically conductive metals, and combinations thereof. With regards to suitable electrically conductive polymers and electrically conductive metals, reference is made to the explanations given below. If the first electrode layer and/or the second electrode layer are/is embodied in the form of layer portions which are embodied separately from one another, then the respective layer portions of the first electrode layer and/or second electrode layer independently of one another can comprise an electrically conductive material or consist of an electrically conductive material selected from the aforementioned group.

In a further configuration of the invention, the first electrode layer and the second electrode layer independently of one another comprise an electrically conductive material or independently of one another consist of an electrically conductive material selected from the group consisting of polypyrrole, doped polyethylene, polyaniline, polythiophene, preferably poly(3,4-ethylenedioxythiophene), gold, platinum, indium, tin, copper, silver, preferably nanoscale silver and/or microscale silver, and combinations of at least two of the aforementioned electrically conductive materials.

In a further configuration of the invention, the first electrode layer comprises an electrically conductive metal, in particular an electrically conductive and nanoscale metal and/or an electrically conductive and microscale metal, or carbon and the second electrode layer comprises an electrically conductive polymer, or vice versa. Alternatively, the first electrode layer and the second electrode layer can consist of the respective aforementioned electrically conductive materials. In particular, the electrically conductive metal can be selected from the group consisting of silver, preferably nanoscale silver and/or microscale silver, gold, platinum, indium, tin, copper and combinations of at least two of the aforementioned electrically conductive metals. In particular, the electrically conductive polymer can be selected from the group consisting of polythiophene, preferably poly(3,4-ethylenedioxythiophene), polypyrrole, doped polyethylene, polyaniline and combinations of at least two of the aforementioned electrically conductive polymers.

Within the meaning of the present invention, the term “nanoscale metal” should be understood to mean a metal with a particle diameter, in particular a mean particle diameter, of 1 nm to 1000 nm, in particular 10 nm to 500 nm, and by preference 50 nm to 250 nm.

Within the meaning of the present invention, the term “microscale metal” should be understood to mean a metal with a particle diameter, in particular a mean particle diameter, of 1 μm to 100 μm, in particular 3 μm to 50 μm, and by preference 5 μm to 30 μm.

In particular, the first electrode layer and/or the second electrode layer can be produced from a paste containing a nanoscale metal and/or a microscale metal.

In a further configuration of the invention, the electrically conductive metal, in particular the electrically conductive and nanoscale metal and/or the electrically conductive and microscale metal, is silver and the conductive polymer is poly(3,4-ethylenedioxythiophene).

In other words, the first electrode layer comprises silver, in particular nanoscale silver and/or microscale silver, and the second electrode layer comprises poly(3,4-ethylenedioxythiophene), or vice versa, in a further configuration of the invention. In particular, the electrode layers can consist of the respective aforementioned materials. The use of silver, in particular nanoscale silver, is advantageous in that it can be applied, in particular printed on, in particularly fine structures, for example by means of screen printing. For example, the silver can have a diameter, in particular a mean diameter, of 0.5 μm to 30 μm, in particular 1 μm to 20 μm, and by preference 5 μm to 15 μm. The use of poly(3,4-ethylenedioxythiophene) advantageously makes it possible to avoid the case where, in particular, punctures arise during high-voltage polarization in the piezo layer situated between the first electrode layer and the second electrode layer.

Moreover, an electrode layer with or made of poly(3,4-ethylenedioxythiophene) advantageously has a self-healing effect in the case of an electrical puncture by virtue of the layer resealing itself at the affected location on account of the arising generation of heat. Further, the smooth surface of poly(3,4-ethylenedioxythiophene) has a positive effect with regards to the prevention of electrical breakdowns. Overall, this can consequently eliminate the risk of a permanent short circuit between the electrode layers.

The piezo layer can be embodied contiguously in one piece or in the form of layer portions which are embodied separately from one another.

Additionally, the piezo layer can have a layer thickness of 0.5 μm to 30 μm, in particular 1 μm to 20 μm, and by preference 1 μm to 10 μm.

Further, in principle, all piezoresistive materials, in particular piezoresistive polymers, piezoresistive ceramics and combinations thereof, come into consideration for the piezo layer. The piezoresistive polymers can be homopolymers or copolymers, in particular bipolymers and/or terpolymers, in particular as described hereinbelow.

In a further configuration of the invention, the piezo layer comprises a material or the piezo layer consists of a material selected from the group consisting of polyvinylidene fluoride (PVDF), poly(vinylidene fluoride-trifluoroethylene) (P(VDF-TrFE)), poly(vinylidene fluoride-trifluoroethylene-chlorotrifluoroethylene) (P(VDF-TrFE-CTFE)), poly(vinylidene fluoride-trifluoroethylene-chlorofluoroethylene) (P(VDF-TrFE-CFE)), lead zirconate titanate (PZT), lead magnesium niobate (PMN), zinc oxide (ZnO), aluminum nitride (AlN) and combinations of at least two of the aforementioned materials. If the piezo layer is embodied in the form of layer portions which are embodied separately from one another, then the layer portions independently of one another can comprise a material or consist of a material selected from the aforementioned group.

By preference, the piezo layer comprises poly(vinylidene fluoride-trifluoroethylene) (P(VDF-TrFE)) or consists of poly(vinylidene fluoride-trifluoroethylene) (P(VDF-TrFE)).

Further, the piezo layer can be contiguously applied, in particular printed on, in the circumferential direction of the distal end of the medical instrument. By contrast, the remaining functional layers can be applied to, in particular printed on, the distal end of the medical instrument only in sections. Advantageously, this allows a uniform capture of measured variables in the circumferential direction. As a result, the capture of measured variables is independent of an angular alignment of the distal end of the medical instrument, for example vis-à-vis an ultrasonic source. As a result, it is possible to obtain a yet further improved capture of measured variables and, in particular, an improved localization, for example ultrasound-based localization, of the medical instrument.

Further, the first conductor track, in particular only the first conductor track, can be embodied contiguously in one piece or in the form of layer portions which are embodied separately from one another. Further, the second conductor track, in particular only the second conductor track, can be embodied contiguously in one piece or in the form of layer portions which are embodied separately from one another.

Further, the first conductor track can be embodied contiguously in one piece, and the second conductor track can be embodied in the form of layer portions which are embodied separately from one another. Alternatively, the first conductor track can be embodied in the form of layer portions which are embodied separately from one another, and the second conductor track can be embodied contiguously in one piece. Alternatively, the first conductor track and the second conductor track can each be embodied contiguously in one piece or each be embodied in the form of layer portions which are embodied separately from one another.

Further, the first conductor track can have a layer thickness of 0.1 μm to 30 μm, in particular 0.5 μm to 10 μm, and by preference 1 μm to 2 μm. The second conductor track can further have a layer thickness of 0.1 μm to 30 μm, in particular 0.5 μm to 10 μm, and by preference 1 μm to 2 μm.

Further, the first conductor track and the second conductor track can be embodied differently, in particular at least in portions, for example only in portions or throughout.

Alternatively, the first conductor track and the second conductor track can be embodied the same, in particular at least in portions, for example only in portions or throughout.

In principle, any conductive material is suitable for the first conductor track and the second conductor track. However, conductive materials with an electrical resistivity that is as low as possible, with a certain amount of flexibility and with good adhesive properties, in particular on the insulation layer of the second portion, are preferred.

In a further configuration of the invention, the first conductor track and the second conductor track each comprise an electrically conductive metal, in particular an electrically conductive and nanoscale metal and/or an electrically conductive and microscale metal. In particular, the first conductor track and the second conductor track can each consist of such a metal.

By preference, the first conductor track and the second conductor track independently of one another comprise an electrically conductive metal, in particular silver, preferably nanoscale silver and/or microscale silver, gold, platinum, indium, tin, copper or combinations of at least two of the aforementioned metals. If the first conductor track and/or the second conductor track are/is embodied in the form of layer portions which are embodied separately from one another, then the respective layer portions of the first conductor track and/or second conductor track independently of one another can comprise a material or consist of a material selected from the aforementioned group.

By preference, the first conductor track and/or the second conductor track comprise/comprises silver, in particular nanoscale silver. In particular, the first conductor track and/or the second conductor track can consist of silver, in particular nanoscale silver. By preference, the silver has a diameter, in particular a mean diameter, of 1 nm to 1000 nm, in particular 10 nm to 500 nm, and by preference 50 nm to 250 nm. Correspondingly embodied conductor tracks advantageously have reduced brittleness and increased pliability or flexibility. A further advantage consists in the fact that, in the case of silver, the first conductor track and the second conductor track adhere better to a functional layer, in particular insulation layer, located below the conductor tracks in relation to a layer thickness direction D of the sensor stack.

In a further configuration of the invention, the sensor stack, in particular the first portion and/or second portion of the sensor stack, further comprises an adhesion layer. The adhesion layer preferably serves for integrally bonding the sensor stack to the medical instrument, in particular by way of adhesive bonding. By preference, the adhesion layer is arranged, in particular directly or not directly, between the first insulation layer of the first portion and the surface of the medical instrument and/or, in particular directly or not directly, between the insulation layer of the second portion and the surface of the medical instrument.

In particular, the first portion of the sensor stack can comprise an adhesion layer which is arranged, in particular directly or not directly, between the first insulation layer and the surface of the medical instrument.

By preference, the adhesion layer is arranged, in particular directly or not directly, below the first insulation layer of the first portion in relation to a layer thickness direction D of the sensor stack.

Further preferably, the adhesion layer of the first portion is directly applied to, in particular printed on, the medical instrument.

Further, the second portion of the sensor stack in particular can (also) comprise an adhesion layer which is arranged, in particular directly or not directly, between the insulation layer and the surface of the medical instrument.

By preference, the adhesion layer is arranged, in particular directly or not directly, below the insulation layer of the second portion in relation to a layer thickness direction D of the sensor stack.

Further preferably, the adhesion layer of the second portion is (also) directly applied to, in particular printed on, the medical instrument.

Further, the adhesion layer described in the preceding paragraphs can be embodied contiguously in one piece or in the form of layer portions which are embodied separately from one another.

Further, the adhesion layer of the first portion, in particular only of the first portion, can be embodied contiguously in one piece or in the form of layer portions which are embodied separately from one another. Further, the adhesion layer, in particular only of the second portion, can be embodied contiguously in one piece or in the form of layer portions which are embodied separately from one another.

Further, the adhesion layer of the first portion can be embodied contiguously in one piece, and the adhesion layer of the second portion can be embodied in the form of layer portions which are embodied separately from one another. Alternatively, the adhesion layer of the first portion can be embodied in the form of layer portions which are embodied separately from one another, and the adhesion layer of the second portion can be embodied contiguously in one piece. Alternatively, the adhesion layer of the first portion and the adhesion layer of the second portion can each be embodied contiguously in one piece or each be embodied in the form of layer portions which are embodied separately from one another.

Further, the adhesion layer of the first portion and the adhesion layer of the second portion can be embodied contiguously in one piece.

Alternatively, the adhesion layer of the first portion can be applied, in particular printed on, separately from the adhesion layer of the second portion and can be integrally bonded to the latter.

Further, the adhesion layer, in particular of the first portion and/or second portion, can have a layer thickness of 1 μm to 50 μm, in particular 2 μm to 30 μm, and by preference 5 μm to 15 μm.

In a further configuration of the invention, the adhesion layer, in particular of the first portion and/or second portion of the sensor stack, comprises an electrically conductive metal, in particular an electrically conductive and microscale metal. By preference, the metal is selected from the group consisting of silver, gold, platinum, indium, tin, copper and combinations of at least two of the aforementioned metals. If the adhesion layer is embodied in the form of layer portions which are embodied separately from one another, then the layer portions independently of one another can comprise a metal or consist of a metal selected from the aforementioned group.

In a further configuration of the invention, the metal of the adhesion layer, in particular of the first portion and/or second portion, is silver, in particular microscale silver. By preference, the silver has a diameter, in particular a mean diameter, of 0.5 μm to 30 μm, in particular 1 μm to 20 μm, and by preference 5 μm to 15 μm. A correspondingly configured adhesion layer is advantageously distinguished by reduced brittleness and increased pliability or flexibility.

In a further configuration of the invention, the sensor stack, in particular the first portion and/or second portion, preferably the second portion, of the sensor stack, further comprises an anticorrosion layer. This can advantageously ensure protection of the conductor tracks from corrosion, in particular in the region of contact with other materials, during the signal transfer and safeguard a long-term constant electrical transfer resistance and low contact resistance.

By preference, the anticorrosion layer is in particular directly or not directly applied, in particular printed on, above the first conductor track and the second conductor track in relation to a layer thickness direction D of the sensor stack.

The anticorrosion layer can be embodied contiguously in one piece. Alternatively, the anticorrosion layer can be embodied in the form of layer portions which are embodied separately from one another.

Additionally, the anticorrosion layer can have a layer thickness of 0.5 μm to 30 μm, in particular 1 μm to 20 μm, and by preference 2 μm to 10 μm.

In principle, all non-corroding and conductive materials are suitable for the anticorrosion layer.

In a further embodiment of the invention, the anticorrosion layer comprises a material or consists of a material selected from the group consisting of carbon, noble metals, in particular gold and/or platinum, and combinations of at least two of the aforementioned materials. If the anticorrosion layer is embodied in the form of layer portions which are embodied separately from one another, then the layer portions independently of one another can comprise a material or consist of a material selected from the aforementioned group.

By preference, the anticorrosion layer comprises conductive carbon. In particular, the anticorrosion layer can consist of conductive carbon. Carbon was found to be particularly advantageous in respect of avoiding corrosion and hence ensuring an electrical transfer resistance which is constant over time. Moreover, carbon is for example softer than the aforementioned noble metals and can therefore better cling to a contact.

In a further configuration of the invention, the sensor stack, in particular the first portion and/or second portion, in particular only the first portion, of the sensor stack, comprises a biocompatible capping layer. By preference, the capping layer is arranged on the outside in relation to a layer thickness direction D of the sensor stack. By preference, the capping layer is applied to, in particular printed on, in particular directly or not directly, above the shielding layer in relation to a layer thickness direction D of the sensor stack. Accordingly, the biocompatible capping layer can preferably form a (terminating) outer layer of the sensor stack, in particular of the first portion of the sensor stack.

Direct contact of the remaining functional layers with the body of the patient is advantageously prevented by way of the biocompatible capping layer. This counteracts possible undesirable chemical and/or biological interactions, and hence a possible adverse effect on the health of the patient.

The biocompatible capping layer can be embodied contiguously in one piece. Alternatively, the biocompatible capping layer can be embodied in the form of layer portions which are embodied separately from one another.

Additionally, the biocompatible capping layer can have a layer thickness of 1 μm to 30 μm, in particular 2 μm to 20 μm, and by preference 5 μm to 15 μm.

In principle, all biocompatible and in particular electrically insulating materials are suitable for the biocompatible capping layer. By preference, the biocompatible capping layer comprises a material or consists of a material selecting from the group consisting of polyethylene terephthalate (PET), polyethylene (PE), polytetrafluoroethylene (PTFE), Teflon (PTFE) polyester, parylene, polyester and combinations of at least two of the aforementioned materials. If the capping layer is embodied in the form of layer portions which are embodied separately from one another, then the layer portions independently of one another can comprise a material or consist of a material selected from the aforementioned group.

The first insulation layer of the first portion of the sensor stack can be embodied contiguously in one piece or in the form of a plurality of layer portions which are embodied separately from one another.

Further, the first insulation layer of the first portion and/or the insulation layer of the second portion of the sensor stack can have a layer thickness of 1 μm to 50 μm, in particular 2 μm to 30 μm, and by preference 4 μm to 20 μm.

Further, the second insulation layer of the first portion of the sensor stack can be embodied contiguously in one piece or in the form of a plurality of layer portions which are embodied separately from one another.

Additionally, the second insulation layer of the first portion can have a layer thickness of 1 μm to 50 μm, in particular 5 μm to 40 μm, and by preference 10 μm to 30 μm.

Further, the insulation layer of the second portion of the sensor stack can be embodied contiguously in one piece or in the form of a plurality of layer portions which are embodied separately from one another.

Additionally, the insulation layer of the second portion can have a layer thickness of 1 μm to 50 μm, in particular 5 μm to 40 μm, and by preference 10 μm to 30 μm.

Further, the first insulation layer and the second insulation layer of the first portion and the insulation layer of the second portion can be embodied differently, in particular at least in portions, for example only in portions or throughout.

Alternatively, the first insulation layer and the second insulation layer of the first portion and the insulation layer of the second portion can be embodied the same, in particular at least in portions, for example only in portions or throughout.

In principle, all nonconductive materials are suitable for the first insulation layer and the second insulation layer of the first portion and the insulation layer of the second portion. In particular, the nonconductive materials in this case can be

UV curing materials and/or thermally curing materials. These materials have the advantage of curing quickly and thus reduce the time required to produce the sensor stack and in particular the medical instrument.

In a further configuration of the invention, the first insulation layer and the second insulation layer of the first portion and the insulation layer of the second portion independently of one another comprise an electrically insulating material selected from the group consisting of UV-curing materials, in particular polyester resins, thermally curing material, in particular as mentioned above, i.e. resins or polyester, plastics, in particular polyethylene terephthalate (PET), polyethylene naphthalate (PEN), polyether sulfone (PES), polysulfone (PSU), polyimide (PI), polycarbonate (PC) or polyetherimide (PEI) and combinations of at least two of the aforementioned electrically insulating materials. If the first insulation layer and/or the second insulation layer of the first portion and/or the insulation layer of the second portion are/is embodied in the form of layer portions which are embodied separately from one another, then the respective layer portions of the first insulation layer and/or the second insulation layer of the first portion and/or the insulation layer of the second portion independently of one another can comprise a material or consist of a material selected from the aforementioned group.

By preference, the first insulation layer of the first portion and the insulation layer of the second portion comprise the same material or consist of the same material at least in portions, in particular only in portions or throughout. By preference, the material is polyethylene naphthalate (PEN). As a result, a high temperature resistance and, in particular, a good adherence are advantageously obtainable for the printing process. In respect of materials that might be considered alternatively, reference is made to the explanations given above in respect of the insulation layers.

Further, the second insulation layer of the first portion and/or the insulation layer of the second portion preferably comprise/comprises a UV-curing material, preferably a polyester resin, or consist/consists of such material.

The use of UV-curing materials, in particular polyester resin, for the embodiment of the second insulation layer of the first portion and/or the insulation layer of the second portion was found to be particularly advantageous with regards to reducing capacitive effects and protecting the functional layers, in particular the electrode layers and conductor tracks, from breaking. This advantageously allows the sensor unit to bend, in particular during the application to a curved surface of the medical instrument, without loss of integrity in the functional layers, in particular in the electrode layers and/or the conductor tracks. A further advantage consists in an improved adhesive effect for directly adjacent functional layers, in particular the shielding layer of the first portion. Moreover, UV-curing materials, in particular polyester resin, do not lead to any interactions with underlying functional layers directly after application, in particular printing, whereby the sensor properties are not adversely affected.

Further, the second insulation layer of the first portion serves as an electrically insulating layer between the shielding layer and the electrode layers of the first portion, in particular the second electrode layer. Particularly advantageously, the distance between the shielding layer and the electrode layers, possible capacitances and the size of the sensor unit can be purposefully influenced by the thickness of the second insulation layer of the first portion.

For example, the second insulation layer of the first portion can have a layer thickness of 1 μm to 50 μm, in particular 5 μm to 40 μm, and by preference 10 μm to 30 μm.

Further, the second insulation layer can be embodied as a layer which is contiguous in one piece or in the form of a plurality of partial layers, for example two or three partial layers, which are embodied separately from one another.

The shielding layer can be embodied contiguously in one piece or in the form of a plurality of layer portions which are embodied separately from one another.

In principle, all electrically conductive materials are suitable for the electrically-shielding shielding layer. However, electrically conductive materials with an electrical resistivity that is as low as possible and good adherence are preferred.

In a further configuration of the invention, the shielding layer comprises a material, in particular an electrically conductive material, or consists of a material, in particular an electrically conductive material, selected from the group consisting of carbon, conductive polymers, in particular polypyrrole, doped polyethylene, polyaniline or polythiophene, preferably poly(3,4-ethylenedioxythiophene), conductive metals, in particular gold, platinum, indium, tin, copper, or silver, preferably nanoscale silver, and combinations of at least two of the aforementioned materials, in particular electrically conductive materials. If the shielding layer is embodied in the form of a plurality of layer portions which are embodied separately from one another, then the layer portions can comprise a material or consist of a material selected from the aforementioned group.

By preference, the shielding layer comprises a conductive metal, in particular silver.

Further, at least one second applied, in particular printed, sensor stack may be provided at the distal end of the medical instrument, the second sensor stack being arranged at a distance from the sensor stack in the longitudinal direction of the distal end. By preference, the second sensor stack has an identical structure to the sensor stack.

As a result of the arrangement of the second sensor stack in a manner spaced apart in the longitudinal direction, it is possible to ascertain not only the position of the distal end but also its angular alignment, especially in relation to an existing ultrasonic field. Consequently, it is advantageously possible not only to locate the position of the distal end but also to locate the alignment, the latter in particular allowing conclusions to be drawn about a feed direction of the distal end of the medical instrument within the body of the patient. The second sensor stack can be formed separately, in particular printed, and can subsequently be applied to, in particular printed on, the distal end of the medical instrument. Alternatively, the second sensor stack can be applied directly to, in particular printed directly on, the distal end. In view of the functional layers of the second sensor stack (including their arrangement), reference is made to the relevant disclosure in the context of the (first) sensor stack, which applies correspondingly to the second sensor stack; this is done to avoid repetition.

According to a second aspect, the invention relates to a method for producing a medical instrument according to the first aspect of the invention, wherein functional layers are applied to, in particular printed on, the distal end of the medical instrument while forming the sensor stack in the process.

Applying the functional layers to, in particular printing the functional layers on, the distal end of the medical instrument makes it possible in particular to dispense with complicated positioning of a separately produced sensor stack on the distal end and a separate production of a joint between the distal end and the sensor stack. Instead, the functional layers of the sensor stack are applied directly to, in particular printed directly on, a wall portion of the distal end of the medical instrument. Moreover, inclusions of air between the sensor stack and the wall portion can be prevented as a result; this is advantageous both in view of a secure connection of the sensor stack to the distal end of the medical instrument and in view of the measuring sensitivity of the sensor stack.

It is also advantageous with regards to the application of the functional layers to, in particular the printing of the functional layers on, the medical instrument that the use of a carrier layer and/stiffening layer is unnecessary. Instead, a first functional layer in relation to a layer thickness direction D of the sensor stack, in particular the adhesion layer of the first and/or second portion or the first insulation layer of the first portion and/or the insulation layer of the second portion, is applied to, in particular printed on, the medical instrument, and all further functional layers are subsequently applied to, in particular printed on, this first functional layer.

By preference, the sensor stack is embodied by means of a printing method, in particular screen printing. Screen printing is associated with the advantage of providing good long-term process stability at low costs. As an alternative to the screen printing method, the sensor stack can also be embodied by means of other printing methods, for example offset printing, inkjet printing, aerosol jet printing, spray coating or dip coating.

Further, the first electrode layer and/or the second electrode layer of the sensor stack can be produced from a metal-containing paste, in particular a paste containing nanoscale metal, preferably nanoscale silver, and/or microscale metal, preferably microscale silver. Alternatively, a conductive metal, for example gold, platinum, indium, tin or copper, can be applied to a film for the purpose of embodying the first electrode layer and/or the second electrode layer.

Further, the first conductor track and/or the second conductor track of the sensor stack can be produced from a metal-containing paste, in particular a paste containing nanoscale metal, preferably nanoscale silver, and/or microscale metal, preferably microscale silver.

Alternatively, a conductive metal, for example gold, platinum, indium, tin or copper, can be applied to a film and shaped to a suitable line width by structuring and/or etching for the purpose of embodying the first electrode layer and/or second electrode layer and/or first conductor track and/or second conductor track. Further functional layers can subsequently be applied, in particular printed on, or applied by means of thin-film technology (vapor deposition or sputtering).

With regards to further features and advantages of the method, in particular in relation to the medical instrument, the sensor stack and the functional layers, reference is made in full to the explanations given within the scope of the first aspect of the invention. The features and advantages described there, in particular in relation to the medical instrument, the sensor stack and the functional layers, also apply analogously to the method according to the second aspect of the invention.

According to a third aspect, the invention relates to a method for producing a medical instrument according to the first aspect of the invention, wherein the functional layers are produced as a separate sensor stack using a carrier layer and/or stiffening layer, and the separately produced sensor stack is subsequently applied to, in particular printed on, the distal end of the medical instrument, the carrier layer and/or stiffening layer being removed in the process.

Within the meaning of the present invention, the term “carrier layer” should be understood to mean a layer to which all further functional layers are applied, preferably with the exception of the adhesion layer, which is preferably applied on the opposite side to the functional layer side of the carrier layer. The carrier layer can also be referred to as a substrate layer within the meaning of the present invention. The carrier layer is preferably designed like a film. By preference, the carrier layer is embodied to allow printing thereon and, in particular, withstand temperatures in a drying process without significant amounts of shrinkage. In particular, the carrier layer can have a layer thickness of 10 μm to 500 μm, in particular 20 μm to 250 μm, and by preference 50 μm to 150 μm.

The stiffening layer can also be referred to as a liner layer within the meaning of the present invention. The stiffening layer is preferably designed like a film. Further preferably, the stiffening layer has a greater layer thickness than the substrate layer. In particular, the stiffening layer can have a layer thickness which is greater than the layer thickness of the carrier layer by at least a factor of 2. For example, the stiffening layer can have a layer thickness of 10 μm to 500 μm, in particular 20 μm to 250 μm, and by preference 50 μm to 150 μm. As a result, the entire structure is advantageously provided with a necessary stiffness, for example to be processable within the scope of a screen printing process. Further, the stiffening layer can in particular cover an adhesion layer applied to a back side of the carrier layer and thereby prevent unwanted adhesive bonding of the substrate layer.

The stiffening layer may also be coated, for example with a silicone. This can advantageously prevent permanent adhesive bonding between the stiffening layer and the carrier layer.

In principle, the carrier layer and the stiffening layer may comprise a differing material or consist of a differing material.

However, by preference, the carrier layer and the stiffening layer comprise the same material or consist of the same material. In particular, the carrier layer material has the same coefficient of thermal expansion as the stiffening layer material.

In particular, the carrier layer material can be selected from the group consisting of polyethylene naphthalate (PEN), polyethylene terephthalate (PET), polyether sulfone (PES), polysulfone (PSU), polyimide (PI), polycarbonate (PC), polyethyleneimine (PEI) and combinations of at least two of the aforementioned polymers.

In particular, the stiffening layer material can be selected from the group consisting of paper, polyethylene naphthalate (PEN), polyethylene terephthalate (PET), polyether sulfone (PES), polysulfone (PSU), polyimide (PI), polycarbonate (PC), polyethyleneimine (PEI) and combinations of at least two of the aforementioned polymers.

Polyethylene naphthalate (PEN) is particularly preferred as material for the carrier layer and the stiffening layer. Alternatively, polyethylene terephthalate (PET) can be preferred as material for the carrier layer and the stiffening layer.

By preference, the adhesion layer described in detail within the scope of the first aspect of the invention is exposed by the removal of the carrier layer and/or stiffening layer.

With regards to further features and advantages of the method, in particular in relation to the medical instrument, the sensor stack and the functional layers, reference is made in full to the explanations given within the scope of the first and second aspects of the invention. The features and advantages described there, in particular in relation to the medical instrument, the sensor stack and the functional layers, also apply analogously to the method according to the third aspect of the invention.

According to a fourth aspect, the invention relates to a sensor unit.

The sensor unit is configured to capture a measured physical variable and convert the latter into an electrical measurement signal, said sensor unit being in the form of a sensor stack having a first portion and a second portion and constructed in layers.

The first portion and the second portion each comprise a plurality of functional layers arranged one above the other, in particular directly or not directly above one another.

The functional layers are applied to, in particular printed on, one another in layers so as to cover one another at least in portions.

The first portion comprises at least the following functional layers:
a first insulation layer covering and electrically insulating the distal end of the medical instrument at least in portions, in particular only in portions or in full, i.e. throughout or contiguously,
an electrically conductive first electrode layer applied to, in particular printed on, the first insulation layer at least in portions, in particular only in portions or in full, i.e. throughout or contiguously,
a piezoactive piezo layer applied to, in particular printed on, the first electrode layer at least in portions, in particular only in portions or in full, i.e. throughout or contiguously,
an electrically conductive second electrode layer applied to, in particular printed on, the piezo layer at least in portions, in particular only in portions or in full, i.e. throughout or contiguously,
an electrically insulating second insulation layer applied to, in particular printed on, the second electrode layer at least in portions, in particular only in portions or in full, i.e. throughout or contiguously, and
an electrically-shielding shielding layer applied to, in particular printed on, the second insulation layer at least in portions, in particular only in portions or in full, i.e. throughout or contiguously.

The second portion comprises at least the following functional layers:
an insulation layer covering and electrically insulating the distal end of the medical instrument at least in portions, in particular only in portions or in full, i.e. throughout or contiguously,
an electrically conductive first conductor track applied to, in particular printed on, the insulation layer at least in portions, in particular only in portions or in full, i.e. throughout or contiguously, and
an electrically conductive second conductor track applied to, in particular printed on, the insulation layer at least in portions, in particular only in portions or in full, i.e. throughout or contiguously.

With regards to further features and advantages of the sensor unit, reference is made in full to the explanations given within the scope of the description above, in particular with regards to the first aspect of the invention. The features and advantages described there in relation to the sensor unit also apply analogously to the sensor unit according to the fourth aspect of the invention.

### BRIEF DESCRIPTION OF THE DRAWINGS

Further advantages and features of the invention emerge from the following description of preferred exemplary embodiments of the invention which are schematically illustrated with the aid of the drawings.

FIG. 1 shows a schematic partially cut perspective illustration of an embodiment of a medical instrument according to the invention in the region of a distal end,

FIG. 2 shows a schematically simplified cross-sectional illustration of the medical instrument according to FIG. 1 along a cut line II-II according to FIG. 1,

FIG. 3 shows a partially cut schematically simplified longitudinal sectional illustration of the medical instrument according to FIGS. 1 and 2 in the region of the distal end,

FIG. 4 schematically shows a first portion of a sensor stack of the medical instrument according to FIGS. 1 to 3,

FIG. 5 schematically shows a second portion of a sensor stack of the medical instrument according to FIGS. 1 to 3, and

FIG. 6 shows a partially cut schematic side view of a further embodiment of a medical instrument according to the invention, with individual components being depicted in schematically much simplified fashion for the purpose of elucidating a basic structure.

### DETAILED DESCRIPTION

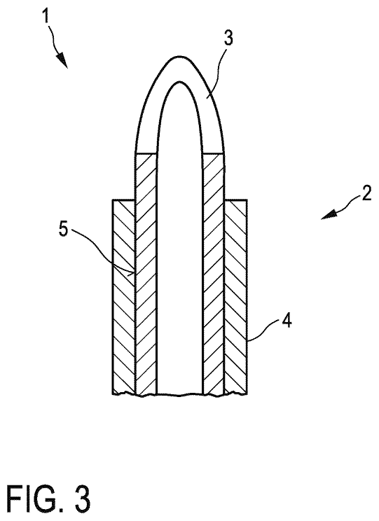

FIG. 1 schematically shows a medical instrument 1, for example in the form of a cannula. Of the medical instrument, only the region of a distal end, provided for insertion into a body of a patient, is visible. For example, the distal end 2 can be a cannula tip which at the end face comprises a puncturing portion 3 embodied in a manner known in principle and whetted at an angle. A proximal end not visible here has a structure known in principle. The medical instrument 1 comprises a sensor unit 4 which is configured to capture a measured physical variable and convert same into electrical measurement signals. The sensor unit 4 is arranged at the distal end 2 and depicted in schematically much simplified fashion, in particular on the basis of FIGS. 1 to 3.

By preference, the sensor unit 4 is embodied in the form of an ultrasonic transducer, as yet to be described in detail, enabling ultrasound-based locating of the tip 2 of the medical instrument 1 in the body of a patient. This allows medical staff to navigate the tip 2 in the body of the patient during a therapeutic and/or diagnostic method, with ultrasound-based methods suitable to this end being known as a matter of principle and also being referred to as tracking. Therefore, tracking as such need not be discussed in more detail here.

The sensor unit is embodied in the form of a sensor stack 4 constructed layer-by-layer. The sensor stack 4 comprises a first portion 4a and a second portion 4b. The portion 4a, which may also be referred to as sensor portion, preferably forms a distal portion of the sensor stack 4. The portion 4b, which may also be referred to as contacting portion, preferably forms a proximal portion of the sensor stack 4.

Further, the first portion 4a and the second portion 4b are preferably arranged in succession, in particular in direct succession. Particularly preferably, the first portion 4a and the second portion 4b are embodied contiguously in one piece. Alternatively, the first portion 4a and the second portion 4b can be embodied separately from one another and can be connected, in particular integrally bonded, to one another.

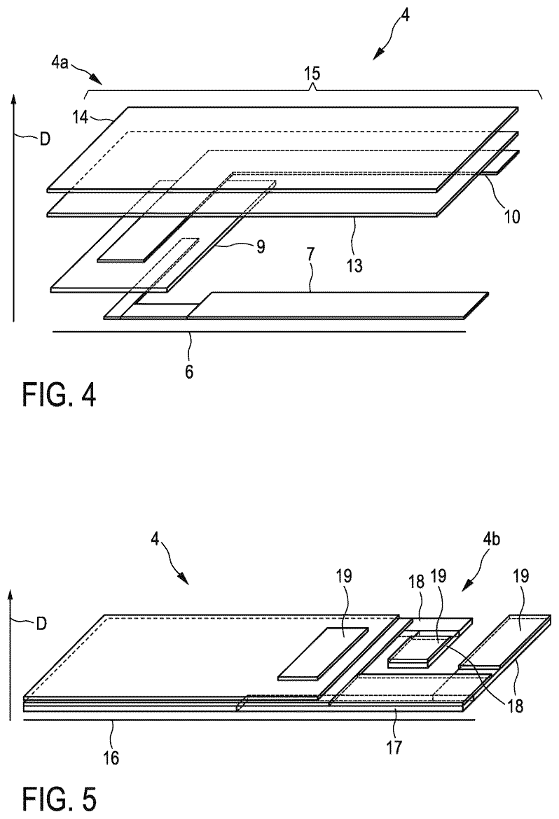

The portion 4a and the portion 4b each comprise a plurality of functional layers arranged one above the other (cf. FIGS. 4 and 5). In this case, the functional layers are applied to, in particular printed on, one another in layers so as to cover one another at least in portions. In this case, the sensor stack 4 is preferably produced by means of a printing method. For example, screen printing techniques, inkjet printing techniques, dispensing printing techniques and/or block printing techniques can be considered as printing methods for producing the sensor stack 4. To embody the individual functional layers of the sensor stack 4, which are yet to be explained in detail, materials with appropriate electrical and/or electronic functional properties, which start off in liquid and/or pasty form, are printed by means of the printing method. The functional layers have not been depicted true to scale. With regard to suitable layer thicknesses, reference is made to the general description. To realize appropriate layer thicknesses, individual materials can be printed on one another multiple times in order to form one of the functional layers.

In this case, the sensor stack 4 is applied directly to, in particular printed directly on, a wall portion 5 of the distal end 2 in the embodiment according to FIGS. 1 to 5, and is consequently securely connected to the distal end 2. As a result, it is possible in particular to manage without a separate application of a sensor stack produced separately at an earlier stage, significantly facilitating the production of the medical instrument 1. Moreover, inclusions of air between the wall portion 5 and the sensor stack 4, as may otherwise occur in the case of conventionally adhesively bonding a sensor stack for example, are prevented.

The layer-by-layer structure of the sensor stack 4 or of its portions 4a and 4b is discussed in more detail below on the basis of FIGS. 4 and 5.

Thus, the first portion 4a of the sensor stack 4 comprises a first insulation layer 6, which comprises an electrically insulating material or is formed from an electrically insulating material. By preference, the first insulation layer 6 is printed directly on the wall portion 5. Accordingly, the first insulation layer 6 preferably forms a lowermost layer of the first portion 4a.

Further, the first portion 4a comprises a first electrode layer 7 which is printed, in particular directly printed, on the insulation layer 6. The first electrode layer 7 comprises an electrically conductive material or is formed from an electrically conductive material.

Further, the first portion 4a comprises a piezoactive piezo layer 9 which is printed, in particular directly printed, on the first electrode layer 7. The piezo layer 9 converts ultrasonic pulses, which are to be captured by preference, into appropriate electrical measurement signals in a manner known in principle. Accordingly, the piezo layer 9 preferably forms an actual ultrasonic transducer of the sensor stack 4, with the remaining functional layers by contrast serving for electrical insulation or conducting away the signal, for example. In particular, the piezo layer 9 can comprise a material or consist of a material selected from the group consisting of polyvinylidene fluoride (PVDF), poly(vinylidene fluoride-trifluoroethylene) (P(VDF-TrFE)), poly(vinylidene fluoride-trifluoroethylene-chlorotrifluoroethylene) (P(VDF-TrFE-CTFE)), poly(vinylidene fluoride-trifluoroethylene-chlorofluoroethylene) (P(VDF-TrFE-CFE)), lead zirconate titanate (PZT), lead magnesium niobate (PMN), zinc oxide (ZnO), aluminum nitride (AlN) and combinations of at least two of the aforementioned materials.

Further, the first portion 4a comprises an electrically conductive second electrode layer 10, which is printed, in particular directly printed, on the piezo layer 9 and which (likewise) comprises an electrically conductive material or is manufactured from an electrically conductive material.

The second electrode layer 10 and the first electrode layer 7 preferably independently of one another comprise an electrically conductive material or preferably independently of one another consist of an electrically conductive material selected from the group consisting of polypyrrole, doped polyethylene, polyaniline, polythiophene, preferably poly(3,4-ethylenedioxythiophene), gold, platinum, indium, tin, copper, silver, preferably nanoscale silver, and combinations of at least two of the aforementioned electrically conductive materials.

By preference, the first electrode layer 7 and the second electrode layer 10 have different embodiments. By preference, the first electrode layer 7 comprises an electrically conductive metal, in particular an electrically conductive and nanoscale metal and/or an electrically conductive and microscale metal, or carbon and the second electrode layer 10 comprises an electrically conductive polymer. Particularly preferable, the metal is silver and the polymer is poly(3,4-ethylenedioxythiophene).

As is further evident from FIG. 4, the first portion 4a moreover comprises a second insulation layer 13, which comprises an electrically insulating material or is formed from an electrically insulating material. The second insulation layer 13 is printed on the second electrode layer 10 and the underlying functional layers. In this case, the second insulation layer 13 is preferably printed directly on the second electrode layer 10. Further, in the region of the electrode layers 7, 10 and the piezo layer 9 in particular, the second insulation layer 13 can be guided around these on the end face side.

The electrically insulating material of the first insulation layer 6 and the electrically insulating material of the second insulation layer 13 can be selected independently of one another from the group consisting of UV-curing materials, in particular polyester resins, thermally curing material, plastics, in particular polyethylene terephthalate (PET), polyethylene naphthalate (PEN), polyether sulfone (PES), polysulfone (PSU), polyimide (PI), polycarbonate (PC) or polyetherimide (PEI) and combinations of at least two of the aforementioned electrically insulating materials.

Moreover, the first portion 4a comprises a shielding layer 14 which is printed, in particular directly printed, on the second insulation layer 13 and which electrically and/or magnetically shields the latter layer and the further functional layers therebelow in a layer thickness direction D. In particular, the shielding layer 14 can serve to shield the piezo layer 9 from electric and/or magnetic fields which could adversely affect capturing, converting and/or conducting away the signal. In particular, the shielding layer 14 can comprise a material or consist of a material selected from the group consisting of carbon, conductive polymers, in particular polypyrrole, doped polyethylene, polyaniline or polythiophene, preferably poly(3,4-ethylenedioxythiophene), conductive metals, in particular gold, platinum, indium, tin, copper, silver, preferably nanoscale silver, and combinations of at least two of the aforementioned materials.

Moreover, the first portion 4a can comprise a biocompatible capping layer 15 which—in relation to the layer thickness direction D—is arranged on the outside. Accordingly, the biocompatible capping layer 15 forms an outer sleeve of the sensor stack 4. The capping layer 15 comprises a biocompatible material or is formed from a biocompatible material. For example, the material can be selected from the group consisting of polyethylene terephthalate (PET), polyethylene (PE) and combinations of at least two of the aforementioned materials. The biocompatible capping layer 15 prevents the remaining functional layers of the sensor stack 4 from coming into contact with the body of the patient, as this might be accompanied by harmful chemical and/or biological interactions.

The second portion 4b of the sensor stack 4 comprises an insulation layer 16.

By preference, the insulation layer 16 is printed directly on the wall portion 5 of the medical instrument 1. Accordingly, the insulation layer 16 forms a lowermost layer of the second portion 4b. The insulation layer 16 and the first insulation layer 6 of the first portion 4a preferably have the same embodiment. In particular, the first insulation layer 6 of the first portion 4a and the insulation layer 16 of the second portion 4b are embodied contiguously in one piece. Alternatively, however, the first insulation layer 6 of the first portion 4a and the insulation layer 16 of the second portion 4b can also be embodied separately from one another and can be connected, in particular integrally bonded, to one another.

Further, an electrically conductive first conductor track 17 which is printed, in particular directly printed, in sections on the insulation layer 16 is provided. The first conductor track 17 can be embodied contiguously in one piece with the first electrode layer 7. Alternatively, the first conductor track 17 can be printed on separately from the first electrode layer 7 and electrically contacted with the latter.

Further, the second portion 4b comprises a second conductor track 18 which is printed, in particular directly printed, in sections on the insulation layer 16 with the second conductor track 18 being printed, in particular directly printed, on another portion of the insulation layer 16.

By preference, the first conductor track 17 and the second conductor track 18 each comprise a conductive metal, in particular a conductive and nanoscale metal, or are formed from such a metal. By preference, the metal is silver. In particular, the first conductor track 17 and the second conductor track 18 can have the same embodiment.

The first conductor track 17 and the second conductor track 18 serve to conduct away the electrical measurement signals from the piezo layer 9 in the direction of a proximal end of the medical instrument 1 (not depicted in detail here). In this case, the conductor tracks 17, 18 are preferably spaced apart from one another and guided on the insulation layer 16 in the direction of the proximal end. Starting from this proximal end, the conductor tracks 17, 18 can be electrically conductively contactable with an evaluation unit for evaluating the electrical measurement signals from the piezo layer 9.

The second portion 4b can also comprise an anticorrosion layer 19.

The anticorrosion layer 19 can be embodied—as illustrated—in the form of layer portions 19a, 19b and 19c which are embodied separately from one another. Alternatively, the anticorrosion layer 19 can also be embodied contiguously in one piece. In relation to the layer thickness direction of the sensor stack 4, the anticorrosion layer 19 is printed on above the first conductor track 17 and the second conductor track 18. For example, the anticorrosion layer 19 can comprise a material or consist of a material selected from the group consisting of carbon, noble metals, in particular gold and/or platinum, and combinations of at least two of the aforementioned materials.

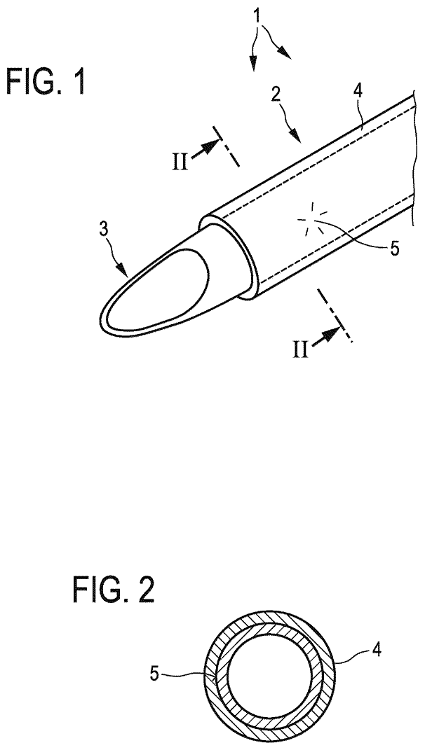

As evident from FIG. 2, the medical instrument 1 has an approximately annular hollow cross section in the present case. By preference, at least the piezoactive piezo layer 9 is printed on contiguously without gaps in the circumferential direction of the distal end 2. Accordingly, the piezo layer 9 preferably encloses the annular wall portion 5, visible in FIG. 2, all round without gaps. In respect of ultrasound-based tracking of the tip 2, this is particularly advantageous inasmuch as a gap-related signal drop is avoided. In the prior art, such a drop in signal is determinable, in particular, whenever a conventional sensor stack is predominantly wound around a cannula tip and adhesively bonded to the cannula tip with the formation of an end face-side gap. A signal drop and hence an adverse effect on the tracking cannot be readily avoided when such a gap is aligned parallel to an ultrasonic incoming radiation direction. This is counteracted by the piezo layer presently printed on contiguously without gaps in the circumferential direction of the distal end 2. In particular, the remaining functional layers of the sensor stack 4, in particular with exception of the conductor tracks 17, 18, can also be printed on all round in the circumferential direction, with the result that the sensor stack 4 has an annular cross section.

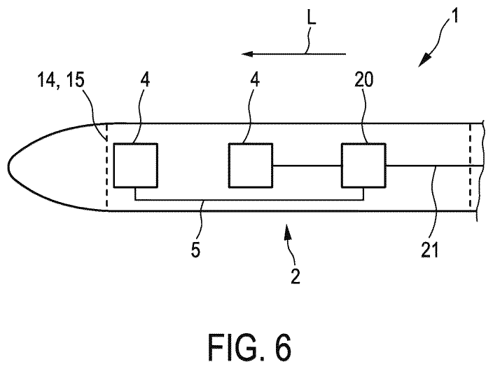

FIG. 6 shows a further embodiment of the medical instrument 1 which substantially corresponds to the embodiment described above on the basis of FIGS. 1 to 5. Thus, reference is made to the disclosure in the context of this embodiment for the purpose of avoiding repetition. Only the essential differences between the embodiment according to FIG. 6 and the previous embodiment are discussed below.

The medical instrument 1 evident from FIG. 6 is depicted in a partially cut and much simplified side view. The medical instrument is preferably embodied in the form of a cannula 1. In contrast to the above-described embodiment, the medical instrument 1 comprises two sensor stacks. These sensor stacks 4 either can be printed directly on the distal end 2 of the medical instrument 1 or can be printed separately and subsequently applied to the distal end 2. Otherwise, the structure of the functional layers of the sensor stack 4 corresponds to the structure of the sensor stack 4 which was described above and depicted in FIGS. 1 to 5. The two sensor stacks 4 are arranged spaced apart from one another in the longitudinal direction L of the distal end 2. For example, an ultrasound-based determination of an alignment of the distal end 2 can be implemented as a result of this type of arrangement. To this end and expressed in simplified terms, each of the two sensor stacks 4 can be “tracked” in ultrasound-based fashion, for example, on the basis of methods known as a matter of principle. If the respective position of both sensor stacks 4 is known as a result, then this allows the alignment of the distal end 2 to be deduced immediately.

Moreover, an amplifier unit 20 arranged at the distal end 2 is provided.

In the present case, the amplifier unit 20 serves to amplify the measurement signals from the two sensor stacks 4. In the case of an embodiment not depicted in detail in the drawings, there might be only one sensor stack and the latter can be electrically conductively connected to the amplifier unit for signal amplification purposes. In the present case, the amplifier unit 20 is electrically conductively connected to both sensor stacks 4 by means of signal lines not labeled in detail and is embodied in the form of a printed circuit. The printed circuit 20 is connectable by means of a further signal line 21 to an evaluation unit for evaluating the amplified measurement signals. In the present case, the printed circuit 20 is printed directly on the wall portion 5 of the distal end 2. In the case of an embodiment not depicted in detail in the drawings, the printed circuit 20 can initially be printed separately and subsequently be applied to the wall portion 5.

As indicated by the two dash-dotted lines in FIG. 6, the two sensor stacks 4, the printed circuit 20 and the signal lines extending between these components are presently covered by means of a metallic shielding layer 14 and/or a biocompatible capping layer 15. With the exception of a puncturing portion of the medical instrument 1 not depicted in detail in the drawings, the shielding layer 14 and/or the capping layer 15 in this case cover the entire distal end 2.

With regards to further features and advantages regarding the embodiments of a medical instrument according to the invention as depicted in FIGS. 1 to 6, reference is made in full to the general description.

1.-16. (canceled)

17. A medical instrument comprising:
a distal end configured for insertion into a patient; and
a sensor unit which arranged at the distal end and configured to capture a measured physical variable and convert the measured physical variable into an electrical measurement signal,
the sensor unit comprising a sensor stack constructed in layers and having a first portion and a second portion, the first portion and the second portion each comprising functional layers arranged one above another, the functional layers being applied to one another in layers so as to cover one another at least in portions,
the first portion comprising:
a first insulation layer covering and electrically insulating the distal end of the medical instrument at least in portions;
a first electrode layer applied to the first insulation layer at least in portions, the first electrode layer being electrically conductive;
a piezo layer applied to the first electrode layer at least in portions, the piezo layer being piezoelectrically active;
a second electrode layer applied to the piezo layer at least in portions, the second electrode layer being electrically conductive;
a second insulation layer applied to the second electrode layer at least in portions, the second insulation layer being electrically insulating; and
a shielding layer applied to the second insulation layer at least in portions, and the second portion comprising:
an insulation layer covering and electrically insulating the distal end of the medical instrument at least in portions;
a first conductor track applied to the insulation layer at least in portions, the first conductor track being electrically conductive; and
a second conductor track directly applied to the insulation layer at least in portions, the second conductor track being electrically conductive.

18. The medical instrument according to claim 17, wherein the first electrode layer and the second electrode layer have different embodiments.

19. The medical instrument according to claim 17, wherein the first electrode layer and the second electrode layer independently of one another comprise an electrically conductive material selected from the group consisting of polypyrrole, doped polyethylene, polyaniline, polythiophene, gold, platinum, indium, tin, copper, silver, and combinations thereof.

20. The medical instrument according to claim 17, wherein the first electrode layer comprises an electrically conductive metal or carbon, and the second electrode layer comprises an electrically conductive polymer.

21. The medical instrument according to claim 20, wherein the electrically conductive metal is silver and the electrically conductive polymer is poly(3,4-ethylenedioxythiophene).

22. The medical instrument according to claim 17, wherein the piezo layer comprises a material selected from the group consisting of polyvinylidene fluoride, poly(vinylidene fluoride-trifluoroethylene), poly(vinylidene fluoride-trifluoroethylene-chlorotrifluoroethylene), poly(vinylidene fluoride-trifluoroethylene-chlorofluoroethylene), lead zirconate titanate, lead magnesium niobate, zinc oxide, aluminum nitride and combinations thereof.

23. The medical instrument according to claim 17, wherein the first conductor track and the second conductor track each comprise an electrically conductive metal.

24. The medical instrument according to claim 23, wherein the electrically conductive metal is silver.

25. The medical instrument according to claim 17, wherein the sensor stack further comprises an adhesion layer arranged between the first insulation layer of the first portion and a surface of the medical instrument and/or between the insulation layer of the second portion and the surface of the medical instrument.

26. The medical instrument according to claim 25, wherein the adhesion layer comprises an electrically conductive metal.

27. The medical instrument according to claim 26, wherein the electrically conductive metal is silver.

28. The medical instrument according to claim 17, wherein the second portion further comprises an anticorrosion layer which is applied above the first conductor track and the second conductor track in relation to a layer thickness direction D of the sensor stack.

29. The medical instrument according to claim 28, wherein the anticorrosion layer comprises a material selected from the group consisting of carbon, noble metals, and combinations thereof.

30. The medical instrument according to claim 17, wherein the sensor stack comprises a biocompatible capping layer arranged on an outside in relation to a layer thickness direction D of the sensor stack.

31. The medical instrument according to claim 17, wherein the first insulation layer and the second insulation layer of the first portion and the insulation layer of the second portion independently of one another comprise an electrically insulating material selected from the group consisting of UV-curing materials, thermally curing materials, plastics, and combinations thereof.

32. The medical instrument according to claim 17, wherein the shielding layer comprises a material selected from the group consisting of carbon, conductive polymers, conductive metals, and combinations thereof.

33. A method for producing a medical instrument according to claim 17, the method comprising the steps of:
applying the functional layers to the distal end of the medical instrument; and
forming the sensor stack as the functional layers are applied to the distal end of the medical instrument.

34. A method for producing a medical instrument according to claim 17, the method comprising the steps of:
producing the functional layers as a separate sensor stack using a carrier layer and/or a stiffening layer; and
applying the sensor stack to the distal end of the medical instrument, with the carrier layer and/or stiffening layer being removed.
